(19) United States
(12) Patent Application Publication
(10) Pub. No.: US 2011/0071348 A1
(43) Pub. Date: Mar. 24, 2011

(54) TROCART

(76) Inventor: Alec Blanchard, Lausanne (CH)

(21) Appl. No.: 12/991,987

(22) PCT Filed: May 5, 2009
(86) PCT No.: PCT/IB2009/005521

§ 371 Date: Nov. 23, 2010

(30) Foreign Application Priority Data
May 14, 2008 (CH) 00733/08

(51) Int. Cl. A61B 1/04; A61B 17/34

(52) U.S. Cl. 600/104; 604/264

(57) ABSTRACT

The invention relates to a trocar including a main tube (1) defined by a proximal end (2) and a distal end (13), comprising a central duct placed between said ends (2,13), and adapted to receive one or more minimally invasive surgery instruments (8); the lateral surface of the main tube (1) comprising at least one opening sized so as to enable placement of one or more other minimally invasive surgery instruments (9,10) transversely to the main tube (1), along a direction forming an angle relative to the main axis thereof, such that the distal end (10) of said other instrument(s) (9,10) is not present in the extension of the main axis of said tube (1).

The present invention relates to minimally invasive surgery, in particular laparoscopic, thoracoscopic, arthroscopic surgery and NOTES (Natural Orifice Trans Endoscopic Surgery) surgery.

It relates most particularly to the trocars used for this purpose.

Laparoscopic surgery makes it possible to carry out many interventions. Usually, this type of surgery is carried out with the aid of several trocars which are placed so that the surgeon obtains a good triangulation between the various trocars in order to have in particular a lateral view of the operation area relative to the operating instruments. For the patient, placing several trocars represents several scars which may be painful and which may be the subject of secondary infection. Moreover, many scars represent an esthetic embarrassment for the patient. To alleviate these drawbacks, technological progress currently makes it possible to carry out an intervention using a single trocar, the trocar comprising several working channels. With NOTES surgery, the natural tracts are used to penetrate the abdominal cavity, for example through the stomach, the rectum or the fornix of the vagina. For this tract, surgeons also use a single trocar comprising several working channels.

Trocars for laparoscopic, thoracoscopic, arthroscopic surgery and NOTES surgery are well known in the surgical industry. These trocars consist of a main tube which may comprise one or more working channels inside the tube. The major drawback of trocars with several working channels is that there is only one axial view of the operation area, which does not allow the surgeon, as is the case when using several trocars, to have a good view of the operation area. With the axial view, the surgeon has no depth of field.

The present invention proposes to remedy the abovementioned problems in particular. It relates to a trocar comprising a main tube defined by a proximal end and by a distal end. The main tube comprises a central duct placed between said ends which is suitable for receiving one or more minimally invasive surgical instruments. Moreover, the lateral face of the main tube comprises at least one orifice having dimensions so as to make it possible to place one or more other minimally invasive surgical instruments across the main tube, in a direction that forms an angle relative to its main axis, so that the distal end of said other nstriument(s) is not in line with the main axis of said tube. The distal end of said other instrument(s) can therefore emerge from the distal end of the main tube.

According to a first embodiment of the invention, the lateral face of the main tube comprises two orifices placed on either side of the main tube, in a direction that forms an angle relative to the main axis of said tube; said orifices having dimensions so as to make it possible to place one or more other minimally invasive surgical instruments across the main tube, in said direction that forms an angle relative to its main axis. In this variant, the distal end of said other instrument(s) therefore emerges at the lateral wall of the main tube.

According to another embodiment of the invention, an optical instrument is placed across the orifices. This configuration offers the surgeon a lateral view of the operation area and a depth of field which increases the safety of the surgical manipulation.

The invention will be better understood below by means of a nonlimiting example illustrated by the following figures:

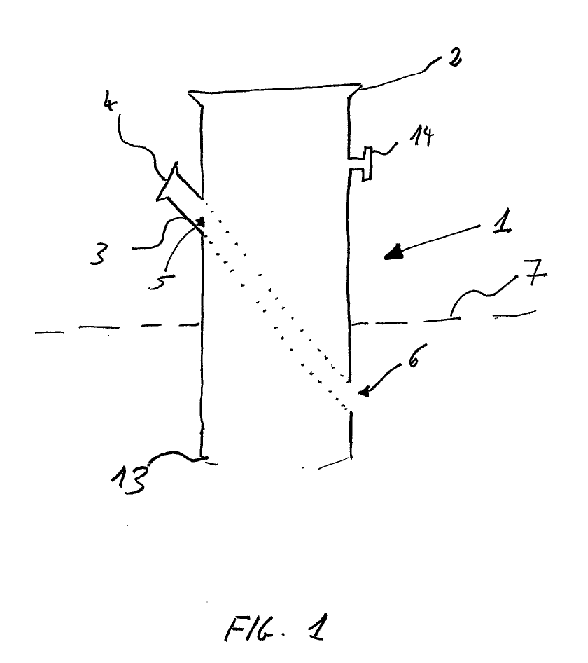

FIG. 1 represents a trocar according to the invention.

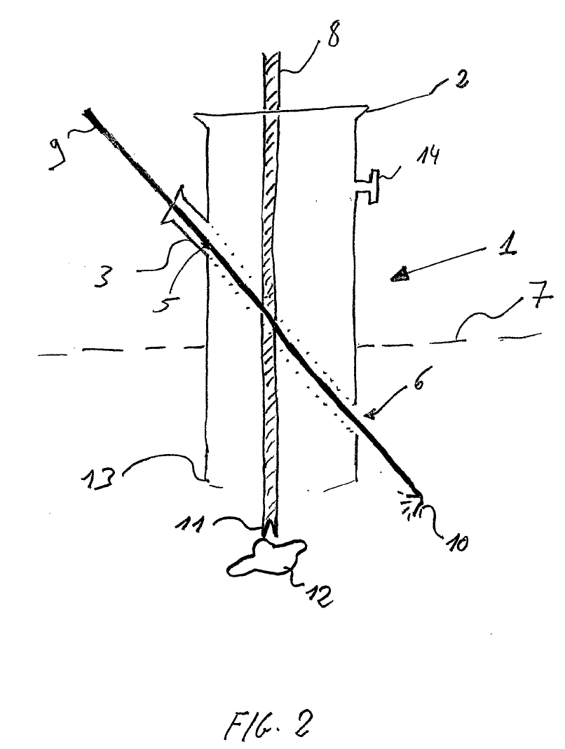

FIG. 2 represents the trocar of FIG. 1 traversed longitudinally by a 1st instrument and obliquely by a 2nd instrument.

The trocar represented schematically in FIGS. 1 and 2 (the dimensional ratios are not representative of reality) comprises a main tube 1 with its working channels and a proximal end 2 comprising a removable valve (not illustrated). As illustrated in FIG. 2, a first instrument 8 is placed in the main duct of the tube 1. Its distal end 11 is positioned in the operation area 12. The trocar is characterized by an additional working channel 3 forming an angle relative to the main tube 1. The additional working channel comprises a valve 4. The walls of the working channel 3 stop at the wall of the main tube 1 in position 5; the latter comprises a lateral orifice as on its opposite wall at 6. These orifices 5, 6, the lateral face of the main tube 1 allow an instrument 9, 10 (see FIG. 2) inserted through the working channel 3 to pass through. The dashed horizontal line 7 represents the interface between the inside and the outside of the patient. By using for example an angular camera in the additional working channel 3, the surgeon has a lateral view of the operation area. The main tube also comprises a connection 14 for CO₂ to pass through in order to create and maintain the pneumoperitoneum.

It goes without saying that the invention is not limited to the trocar illustrated in the above examples.

As indicated above, the invention also relates to a main tube with no second orifice 6.

The optical instrument 9, 10 placed in the additional working channel 3 can be replaced by another minimally invasive surgical instrument. It is also possible to place several instruments in the additional working channel.

Finally it will be noted that additional transverse working channels can also be arranged along the main tube 1.

The angle between the main tube 1 and the working channel 3 can be modified if the working channel 3 is articulated at the orifice 5. If the main tube 1 comprises 2 orifices 5 and 6, the orifice 6 is replaced by a longitudinal slot on the main tube 1 to allow the angle to be changed.

1.-6. (canceled)

7. A trocar comprising a main tube defined by a proximal end and by a distal end, comprising a central duct placed between said ends and suitable for receiving one or more minimally invasive surgical instruments; the lateral face of the main tube comprising at least one orifice having dimensions so as to accommodate a camera across the main tube, in a direction that forms an angle relative to its main axis, so that the distal end of the camera is not in line with the main axis of said tube, and so that it offers a lateral view of an operation area that is in line with the main axis of said tube.

8. The trocar as claimed in claim 7, wherein the lateral face of the main tube comprises two orifices placed on either side of the main tube, in a direction that forms an angle relative to the main axis of said tube; said orifices having dimensions so as to make it possible to place said camera across the main tube, in said direction that forms an angle relative to its main axis.

9. The trocar as claimed in claim 8, wherein said orifices are placed so as to communicate respectively with the outside and the inside of the patient once the trocar is put in place.

10. An assembly formed by a trocar as claimed in claim 7 and a camera, said camera passing through said tube in a direction forming an angle relative to its main axis.

11. The assembly as claimed in claim 10, wherein the camera comprises a distal end forming an angle with the main axis of said instrument, so as to offer a lateral view of an operation area that is in line with the main axis of said tube.

12. The assembly as claimed in claim 11, wherein the camera is angled.
